(19) United States
(12) Patent Application Publication
(10) Pub. No.: US 2007/0128648 A1
(43) Pub. Date: Jun. 7, 2007

(54) Methods for the Identification of IKKalpha Function and other Genes Useful for Treatment of Inflammatory Diseases

(76) Inventors: Jun Li, Danbury, CT (US); Adedeyo Hanidu, Bethel, CT (US); Xiang Li, Danbury, CT (US); Gregory Whitten Peet, Sherman, CT (US); Sheenah Mische, Ridgefield, CT (US); Kenneth Marcu, Stony Brook, NY (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(21) Appl. No.: 11/621,029

(22) Filed: Jan. 8, 2007

Related U.S. Application Data
(63) Continuation of application No. 10/446,045, filed on May 23, 2003, now abandoned.
(60) Provisional application No. 60/406,935, filed on Aug. 29, 2002.
(60) Provisional application No. 60/383,018, filed on May 24, 2002.

(51) Int. Cl. A61K 38/55; C12Q 1/68

(52) U.S. Cl. 435/6; 514/2

(57) ABSTRACT

The invention provides a method for identifying genes involved in the NF-κB pathway comprised of the steps of determining the level of expression of a gene in an experimental sample obtained from the cells having deficient levels of a component of the NF-κB pathway; determining the level of expression of said gene in a control sample obtained from wild type cells having levels of a component of a biological pathway; selecting genes having a level of expression that are modulated in said experimental sample relative to said wild type sample. The invention also provides a method of treating inflammatory related diseases by modulating the activity of IKKα.

### RELATED APPLICATIONS

This application is a Continuation of U.S. Ser. No. 10/446,045, filed May 23, 2003, which claims benefit of U.S. Ser. No. 60/383,018, filed May 24, 2002 and U.S. Ser. No. 60/406,935 filed Aug. 29, 2002; which are hereby incorporated by reference in their entirety.

### BACKGROUND OF THE INVENTION

1. Technical Field

The field of this invention relates to methods and compositions used for the identification and validation of genes involved in biological pathways such as NF-κB useful in the study and treatment of inflammatory disease and cancer.

2. Background Information

Key biological processes such as cell metabolism, cell cycle control, DNA repair and the immune response are known to operate through complex biological pathways that involve the interaction of many genes. Abnormalities in the function of individual genes can in turn alter the function of the biological pathways and often be the cause of disease. In the case of diseases that involve abnormalities in the biological pathways such genes may be suitable for use as novel targets for therapeutic intervention. Accordingly, the identification of genes and the roles they play in biological pathways is of use to modern medicine. An example of a complex biological pathway that is implicated in disease is the NF-κB pathway. The NF-κB or nuclear factor κB is a transcription factor that plays a role in inflammatory diseases by inducing the expression of a large number of pro-inflammatory and anti-apoptotic genes. These include cytokines such as IL-1, IL-2, TNFα and IL-6, chemokines including IL-8 and RANTES, as well as other pro-inflammatory molecules including COX-2 and cell adhesion molecules such as ICAM-1, VCAM-1, and E-selectin. Under resting conditions, NF-κB is present in the cytosol of cells as a complex with IκB. The IκB family of proteins serve as inhibitors of NF-κB, interfering with the function of its nuclear localization signal (see for example U. Siebenlist et al. (1994) Ann Rev Cell Biol 10, 405). Upon disruption of the IκB-NF-κB complex following cell activation, NF-κB translocates to the nucleus and activates gene transcription. Disruption of the IκB-NF-κB complex and subsequent activation of NF-κB is initiated by degradation of IκB.

The NF-κB family includes homo- and heterodimeric transcription factors composed of members of the Rel family (see for example P.A. Baeurle and D. Baltimore. (1996) Cell 87, 13). NF-κB transcription factors bind to DNA as hetero- or homodimers that are selectively derived from five possible subunits (RelA/p65, c-Rel, RelB, p50 and p52) with each binding to half of a conserved 10 base pair consensus sequence (GGGRNWTYCC). While the RelA/p65 and p50 subunits are ubiquitously expressed, the p52, c-Rel and RelB subunits are more functionally important in specific differentiated cell types (Baldwin, A. , Jr. (1996) Annu Rev Immunol 14, 649-683; Liou, H. C. et al. (1994) Mol Cell Biol 14, 5349-5359). Cytoplasmic p65/p50 heterodimers, c-Rel homodimers and RelB are bound to IκBs (inhibitors of NF-κB) thereby sequestering them in the cytoplasm of most cells that are not experiencing a stress-like response (Baldwin, A. , Jr. (1996) Annu Rev Immunol 14, 649-683; Ghosh, S. et al. (1998) Annu Rev Immunol 16, 225-260).

Activators of NF-κB mediate the site-specific phosphorylation of two amino terminal serines in each IκB which makes nearby lysines targets for ubiquitination thereby resulting in IκB proteasomal destruction. NF-κB is then free to translocate to the nucleus and bind DNA leading to the activation of a host of inflammatory response target genes. (Baldwin, A. , Jr. (1996) Annu Rev Immunol 14, 649-683; Ghosh, S. et al. (1998) Annu Rev Immunol 16, 225-260). Recent evidence has shown that NF-κB subunits dynamically shuttle between the cytoplasm and the nucleus but a dominant acting nuclear export signal in IκBα ensures their transport back to the cytoplasm. It was recently shown that nuclear retention of RefA/p65 is regulated by reversible acetylation with its acetylated form being severely compromised in its ability to interact with IκBα (Chen, L. F. et al. (2001) Science 293, 1653-1657).

In contrast to RelA/p65, c-Rel and RelB, the NF-κB p50 and p52 subunits are derived from p105 and p100 precursor proteins by removal of carboxy-terminal IκB domains, which possess the inhibitory properties of IκBS, with the processing of these precursor proteins being initiated by signal induced phosphorylation. Even though NF-κB is largely considered to be a transcriptional activator, under certain circumstances it can also be directly involved in repressing gene expression {reviewed in (Baldwin, A. , Jr. (1996) Annu Rev Immunol 14, 649-683; Ghosh, S. et al. (1998) Annu Rev Immunol 16, 225-260)}. In the latter scenario, direct repression can result if activation domain deficient homodimers of the NF-κB p50 and p52 subunits bind to NF-κB target sequences instead of activating p50/p65 heterodimers (Kang, S. M. et al. (1992) Science 256, 1452-1456; Plaksin, D. et al. (1993) J Exp Med 177, 1651-1662;

Brown, A. et al. (1994) Mol Cell Biol 14, 2926-2935). The IκB homologue Bcl-3, an abundant nuclear IκB-like protein that is not degraded by NF-κB activating pathways, has been reported to have diverse effects on the binding of p50 or p52 homodimers to DNA depending on its state of phosphorylation, concentration and association with nuclear cofactors (Wulczyn, F. G. et al. (1992) Nature 358, 597-599; Bours, V. et al. (1993) Cell 72, 729-739; Nolan, G. P. et al. (1993) Mol Cell Biol 13, 3557-3566; Dechend, R. et al. (1999) Oncogene 18, 3316-3323). Bcl-3 readily forms ternary complexes with DNA bound p50 and p52 homodimers and in that context functions like a transcriptional activator, with its activation potential enhanced by interaction with the Tip60 histone acetylase (Bours, V. et al. (1993) Cell 72, 729-739; Dechend, R. et al. (1999) Oncogene 18, 3316-3323; Fujita, T. et al. (1993) Genes Dev 7, 1354-1363; Pan, J. et al. (1995) J Biol Chem 270, 23077-23083; Hirano, F. et al. (1998) Mol Cell Biol 18, 1266-1274). Complexes of Bcl-3/p50 homodimers were recently shown to contribute to the transcriptional activation of the survival promoting Bcl-2 NF-κB target gene (Kurland, J. F. et al. (2001) J Biol Chem 276, 45380-45386). Bcl-3-p50 complexes form with the same kinetics as p50-p65 heterodimers but are independent of p50-p65 release from IκBα also implicating a p105 proteolysis pathway in their production (Heissmeyer, V. et al. (1999) Embo J 18, 4766-4778).

The phosphorylation of IkB is a major triggering event in regulation of the NF-kB pathway. Since the abnormal regulation of the NF-kB pathway is thought to correlate with inflammatory disease the regulation of IkB phosphorylation would be an important area for disease intervention.

The search for the kinase responsible for the inducible phosphorylation of IκB has been one of the major focuses in the NF-kB field. IkB phosphorylation is mediated by a high molecular weight signalsome complex consisting of at least three components or subunits: two IκB kinases: IKKα, IKKβ and a non-catalytic regulatory subunit NEMO (henceforth, collectively referred to as the signalsome){reviewed in (Mercurio, F. et al. (1999) Oncogene 18, 6163-6171; Barkett, M. et al. (1999) Oncogene 18, 6910-6924; Karin, M. (1999) Oncogene 18, 6867-6874)}. A great deal of work has been performed to determine the respective roles each of the components play in the regulation of NF-kB with the belief that a greater understanding of the roles might lead to the development of new methods and approaches for the treatment of inflammatory diseases. Two molecules of NEMO are believed to orchestrate the assembly of the IKK's into the high molecular weight signalsome complex at least in part by binding to specific carboxy-terminally conserved residues of both IKKα and IKKβ termed the NEMO binding domain or NBD (Krappmann, D. et al. (2000) J Biol Chem 275, 29779-29787; Li, X. H. et al. (2001) J Biol Chem 276, 4494-4500; Hatada, E. N. et al. (2000) Current Opinion in Immunology 12, 52-58; May, M. J. et al. (2000) Science 289, 1550-1554). NEMO may also facilitate the recruitment of IκBα to the IKK complex (Yamamoto, Y. et al. (2001) J Biol Chem 276, 36327-36336). The two catalytic IKK subunits differentially respond via NEMO to an array of signal induced, upstream kinase activities culminating in the coordinated phosphorylation of a pair of serines in their MAPK-like T activation loops by an unknown mechanism.

The roles of the IKKs in NF-κB activation were studied in mice lacking IKKβ, IKKα or NEMO (Li, Q. et al. (1999) Science 284, 321-325; Li, Z. W. et al. (1999) J Exp Med 189, 1839-1845; Tanaka, M. et al. (1999) Immunity 10, 421-429; Li, Q. et al. (1999) Genes Dev 13, 1322-1328; Hu, Y. et al. (1999) Science 284, 316-320; Takeda, K. et al. (1999) Science 284, 313-316). Akin to mice genetically deficient for the NF-κB p65 subunit (Beg, A. A. et al. (1995) Nature 376, 167-170), murine embryos genetically null for either IKKβ or NEMO succumbed to severe liver apoptosis in utero due to a virtually complete block in NF-κB activation (Li, Q. et al. (1999) Science 284, 321-325; Li, Z. W. et al. (1999) J Exp Med 189, 1839-1845; Tanaka, M. et al. (1999) Immunity 10, 421-429; Rudolph, D. et al. (2000) Genes and Dev. 14, 854-862; Schmidt-Supprian, M. et al. (2000) Mol Cell 5, 981-992; Makris, C. et al. (2000) Mol Cell 5, 969-979). These IKKβ and NEMO knockout (KO) animals were severely if not completely deficient for both cytokine mediated IκB degradation and nuclear NF-κB DNA binding activity (Li, Q. et al. (1999) Science 284, 321-325; Li, Z. W. et al. (1999) J Exp Med 189, 1839-1845; Tanaka, M. et al. (1999) Immunity 10, 421-429; Rudolph, D. et al. (2000) Genes and Dev 14, 854-862; Schmidt-Supprian, M. et al. (2000) Mol Cell 5, 981-992; Makris, C. et al. (2000) Mol Cell 5, 969-979).

In contrast to the IKKβ and NEMO KO mice, IKKα null animals died perinatally due to severe skin, limb and skeletal abnormalities caused by a block in the terminal differentiation of epidermal kerotinocytes (Li, Q. et al. (1999) Genes Dev 13, 1322-1328; Hu, Y. et al. (1999) Science 284, 316-320; Takeda, K. et al. (1999) Science 284, 313-316). Subsequent work revealed that IKKα (independent of both its kinase activity and NF-κB), controls the production of a soluble factor that induces kerotinocyte differentiation (Hu, Y., Baud, V. et al. (2001) Nature 410, 710-714). Furthermore, IKKα null embryos appeared to be phenotypically normal for both cytokine induced IκBα degradation, NF-κB nuclear translocation and NF-κB DNA binding activity (Hu, Y. et al. (1999) Science 284, 316-320; Takeda, K. et al. (1999) Science 284, 313-316). In addition, an independent study in cultured mammalian cells, employing transfection conditions that avoided over-expression artifacts, concluded that the cytokine controlled activation of NF-κB induction was an in vivo function of IKKβ and not IKKα (Delhase, M. et al. (1999) Science 284, 309-313).

This body of work has led to the well-accepted belief in the art that IKKβ alone is essential for NF-κB activation by inflammatory response mediators (Karin, M. (1999) Oncogene 18, 6867-6874; Hatada, E. N. et al. (2000) Current Opinion in Immunology 12, 52-58; Karin, M. et al. (2000) Annu Rev Immunol 18, 621-663). However, in spite of this belief reports of inconsistencies with this generally accepted view existed, as two groups have reported some deficiencies in NF-κB transcriptional competence in IKKα (−/−) embryonic fibroblasts (Li, Q., et al Genes and Dev (1999) 1322-1328). More recently and in keeping with its separate and distinct functions from IKKβ, IKKα has been shown to possess at least two additional novel in vivo functions: (a) it is essential for B lymphocyte maturation (Kaisho, T. et al. (2001) J Exp Med 193, 417-426) and Peyers patch formation via an LTβR and NIK dependent signaling pathway (Matsushima, A. et al. (2001) J Exp Med 193, 631-636), wherein it is required to target the cytokine induced processing of the NF-κB2 (p100) precursor to produce the functional NF-κB p52 subunit (Senftleben, U. et al. (2001) Science 293, 1495-1499) and (b) it is required for the proliferation of mammary epithelial cells in response to RANK ligand but not TNFα signaling to activate cyclin D1 (Cao, Y., Bonizzi, G. et al. (2001) Cell 107, 763-775). Independent of these studies, IKKβ was reported to phosphorylate an IκB-like destruction motif in p50's p105 precursor, which produces a recognition site for βTrCP-containing SCF ubiquitin ligases with subsequent polyubiquination of p105 causing its complete proteasomal destruction and the induced release of DNA binding p50 homodimers (Heissmeyer, V. et al. (1999) Embo J 18, 4766-4778; Heissmeyer, V. et al. (2001) Mol Cell Biol 21, 1024-1035), providing additional support for the notion that IKKβ and IKKα have distinct roles in NF-κB activation.

In addition to the well accepted belief of induced nuclear translocation of NF-κB dependent gene expression, an alternative mechanism has emerged that involves the phosphorylation of the p65 transactivation subunit. The protein kinase A catalytic subunit phosphorylates p65 which leads to the association of p65 and the p300 transcriptional coactivator. (Zhong, H. et al (1998) Mol Cell 1, 661-671).

Cells from GSK3 and T2K knockout mice are capable of inducing NF-kB nuclear translocation but are deficient in stimulating transactivation functions of NF-κB (Hoeflich, K.P., et al. (2000) Nature 406, 86-90; Bonnard, et al. (2000) Embo J 19, 4976-4985). IL-1β induces phosphorylation of p65 in an Akt-dependent manner. The ability of Akt to induce transactivation potential of p65 requires IKK and p38 (Madrid, L. V., Mayo, M. W., Reuther, J. Y. and Baldwin, A. S. Jr. (2001) J Biol Chem 276, 18934-18940.) IKKα −/− MEF's, but not IKKβ −/− MEF's are defective in IL-1β mediated p38 activation. This mechanism may partially account for the role of IKKα in NF-kB activated gene transcription.

Methods for the identification and validation of genes involved in biological pathways such as the NF-kB pathway can be used to study diseases and to develop novel targets for disease intervention. Thus, methods for the identification and validation of genes involved in biological pathways such as NF-kB is are considered useful. In addition, methods capable of identifying and validating large numbers of genes involved in such biological pathways (i.e. dozens or hundreds of genes are needed in a single experiment) are considered useful. Furthermore, there is a need for a method of analysis that provides greater understanding of the genes that are involved in the inflammatory response, particularly genes under the influence of the NF-kB pathway. There is also a need for a method for to understanding the roles of the genes that are involved in the NF-kB pathway.

There are limited treatment options available for inflammatory related diseases. Treatments for inflammatory diseases such as asthma include administration of glucocorticoids which directly inhibit activated NF-kB via an interaction between glucocorticoid receptors and NF-kB. There is a need for new methods and approaches for treating inflammatory related diseases.

There is also a need for a method for validating genes that are involved in the inflammatory response because such genes might be suitable for use as targets for therapeutic intervention.

### BRIEF SUMMARY OF THE INVENTION

The present invention is based in part on the applicant's demonstration of the importance of IKKα along with IKKβ and NEMO for activation of the NF-κB dependent genes by employing a method for validating and identifying genes involved in the inflammatory response. Furthermore, this invention is based on the applicant's demonstration that IKKα is also important for the coordinate expression of a host of cellular genes (including mediators of cycle control, DNA repair and apoptosis), whose expression were rescued by blocking NF-κB with a trans-dominant super repressor mutant of IκBα.

One aspect of the present invention relates first to methods for the identification of genes involved in the NF-κB pathway and in particular for those genes under the influence of genes encoding the components of the signalsome complex or the NF-kB pathway including IKKα. Genes identified using the method of the invention can be used as targets for the intervention of immune disease.

One aspect of the present invention is based in part on methods used by the applicant to demonstrate that IKKα, IKKβ and NEMO are each required for the NF-κB mediated inflammatory response program but are differentially involved in NF-κB dependent gene expression.

Another aspect of the invention is the identification of genes useful as therapeutic targets for the treatment of inflammatory diseases, said genes heretofore unknown to be NF-κB dependent genes including several Fox/Forkhead transcription factors, members of the Frizzled family of Wnt signaling receptors, C-EBPβ and C/EBPγ homologous transcriptional regulators of inflammatory responses and SOCS3, a negative effector of STAT3 signaling. NF-κB targeted genes that were also identified using the method of the invention also include genes that are involved in signal transduction, cell cycle and cell proliferation such as G protein coupled receptor RDC1, glucocorticoid-regulated kinase (SGK), phiospholipase D3, hexokinase 2, and Mkp-3/Dual specific protein phosphatase 6.

Another aspect of the invention is a method for identifying genes involved in the NF-κB pathway comprised of the steps of:
a. determining the level of expression of a gene in an experimental sample obtained from the cells having deficient levels of a component of the NF-κB pathway wherein the cells have been exposed to a stimulatory agent;
b. determining the level of expression of said gene in a control sample obtained from wild type cells having wild type levels of said component of the NF-κB pathway wherein the control cells have been exposed to said stimulatory agent;
c. selecting genes having a level of expression that is modulated upward or downward in said experimental sample relative to said wild type sample.

In one aspect of the invention the cells of said experimental sample are deficient in IKKα, IKKβ or NEMO.

In another aspect of the invention the cells of said experimental sample are knockout cells having a null −/− genotype for a component of the signalsome.

In another aspect of the invention the level of gene expression is determined by analysis with a microarray apparatus.

Another embodiment of the invention provides a method for validating target genes:
a. exposing experimental cells deficient in a gene involved in the NF-κB pathway and control cells that are wild type for said gene to a stimulatory agent;
b. determining the level of expression of NF-κB pathway genes implicated;
c. selecting genes as targets for therapeutic intervention if the implicated genes are modulated.

Another aspect of the invention provides for a method of treating inflammatory related diseases by modulating the activity of IKKα.

Another aspect of the invention provides for a method of treating inflammatory disease by modulating the expression of genes that are under the control of IKKα.

### BRIEF DESCRIPTION OF THE FIGURES

FIG. 1 shows the signalsome requirements of selected genes in MEF's that are dependent on NF-κB for their activity.

FIG. 2 shows a hierarchical cluster image of gene expression patterns of NF-κB dependent/TNFα stimulated target genes in MEFs in the presence and absence of individual signalsome subunits.

FIG. 3 shows signalsome subunit requirements of the selected genes for IL-1 dependent signaling.

FIG. 4 shows NF-κB target genes which retain their dependence on IKKα upon prolonged exposure to TNFα.

FIG. 5 shows TaqMan real-time PCR validations of selected induced hits from gene chip screenings.

FIG. 6 shows semi-quantitative RT-PCRs reveal the IKKα and IKKβ requirements of selected MEF genes within 2 hours of TNFα stimulation.

### DETAILED DESCRIPTION OF THE INVENTION

The following definitions are provided to facilitate understanding of terms used herein. Terms not specifically defined herein should be given meanings that would be given to them by one skilled in the art in light of the disclosure and the context.

The term “cells” as used herein includes cells in any form, including but not limited to, cells retained in tissue, cell clusters and individually isolated cells.

The term “cell line” as used herein means a clone of a primary cell line that is capable of stable in vitro growth for many generations.

The term “experimental sample” as used herein is meant an RNA sample from cells that have deficient levels of a component of the NF-κB pathway wherein the cells have been exposed to a stimulatory agent.

The term “control sample” as used herein is meant an RNA sample obtained from cells having wild type activity levels of a component of the NF-κB pathway wherein the cells have been exposed to a stimulatory agent.

The term “gene expression” as used herein means the process by which a gene is converted into an observable phenotype (most commonly production of a protein).

The term “knockout” as used herein means a cell line that does not have a functional copy of the particular gene.

The term “inflammatory agent” as used herein means a compound that is capable of causing an inflammatory response in a cell. “Inflammatory agent” can be used simultaneously with “stimulating agent”.

The term “modulating upward or downward” means the modulation of gene expression by either activating or increasing its expression or repressing or decreasing its expression. Modulations upward or downward are viewed with respect to the level of background inherent in the system used to measure gene expression and the level of gene expression should be distinguishable from the background levels. For instance, if the level of background varies 2 fold a modulation of gene expression of greater than 2 fold would be considered an increase or activation of gene expression. Generally, genes are selected if said gene is modulated upward more than about 2 fold in control relative to wild type levels of expression and preferably more than 5 fold and most preferably more than 10 fold more in control relative to wild type levels of expression. Genes are also selected if said gene is modulated downward more than about 2 fold in control relative to wild type levels of expression and preferably more than about 5 fold and most preferably 10 fold in control relative to wild type levels of expression.

The term “super repressor” means a trans-dominant acting inhibitory protein product or combination of proteins that that have been mutagenically altered to remain in an active state in cells. For instance, the IκB super repressor has been altered so that IκB can not be phosphorylated by the signalsome, thereby retaining its ability to block NF-kB activity even in the context of inflammatory stimuli.

The term “stimulatory agent” means a compound, biological, element or molecule that causes a biological response in a disease mechanism. In the case of NF-κB pathway a stimulatory agent means a compound, element or molecule that causes a NF-κB mediated immune response.

The term “component” means a subunit of the NF-κB signalsome complex. The terms component and subunit can be used interchangeably.

The term “IKKα” as used herein refers to the alpha subunit of the IκB kinase complex. IKKα is a kinase that phosphorylates IκB, NF-κB p100 or other protein substrates.

The term “gene transcription” as it is used herein means a process whereby one strand of a DNA molecule is used as a template for synthesis of a complementary RNA by an RNA polymerase.

The term “DNA” as used herein refers to polynucleotide molecules, segments or sequences and is used herein to refer to a chain of nucleotides, each containing the sugar deoxyribose and one of the four adenine (A), guanine (G) thymine (T) or cytosine (C).

The term “RNA” as used herein refers to polynucleotide molecules, segments or sequences and is used herein to refer to a chain of nucleotides each containing the sugar ribose and one of the four adenine (A), guanine (G) uracil (U) or cytosine (C).

The term “modulating IKKα activity” as used herein means either inhibiting (decreasing) or stimulating (increasing) the level of activity of IKKα protein in a cell. IKKα activity can be modulated by modification of the levels and/or structure of IKKα protein, or by modification of the level of IKKα gene transcription and/or structure such that the levels of IKKα protein activity in the cell is modulated.

The term “protein” as used herein means isolated naturally occurring polypeptides, recombinantly produced proteins. Means for preparing such proteins are well understood in the art. Proteins may be in the form of the secreted protein, including truncated or mature forms. Proteins may optionally be modified to include an additional amino acid sequence which contains secretory or leader sequences, pro-sequences, sequences which aid in purification, such as multiple histidine residues, or an additional sequence for stability during recombinant production. The proteins of the present invention are preferably provided in an isolated form, and preferably are substantially purified. A recombinantly produced version of a protein, including the secreted protein, can be substantially purified using techniques described herein or otherwise known in the art, such as, for example, by the one-step method described in Smith et al, Gene, 67:31-40 (1988). Proteins of the invention also can be purified from natural, synthetic or recombinant sources using techniques described herein or otherwise known in the art.

The term “proinflammatory gene” as used herein refers to any gene that is induced upon an inflammatory response through the NF-κB pathway. Examples of proinflammatory genes include but are not limited to beta inhibin, IL-8, IL-6, interferon stimulated protein, TNF-induced protein, Cox2, GRO1 oncogene, CD44, interleukin 11, and superoxide dismutase. Proinflammatory gene products can be used as stimulatory agents.

Nucleotide sequences are presented herein by a single strand, in the 5′ to 3′ direction, from left to right, using the one letter nucleotide symbols as commonly used in the art and according with the recommendations of the IUPAC-IUB Biochemical Nomenclature Commission (1972).

Methods described herein can be used for assays involved in the study of inflammatory diseases. The term “Inflammatory disease” as used herein could also include autoimmune conditions that involve an inflammatory response such as: osteoarthritis, reperfusion injury, asthma, multiple sclerosis, Guillain-Barre syndrome, Crohn's disease, ulcerative colitis, psoriasis, graft versus host disease, systemic lupus erythematosus, rheumatoid arthritis, toxic shock syndrome, Alzheimer's disease, insulin-dependent diabetes mellitus, acute and chronic pain as well as symptoms of inflammation and cardiovascular disease, stroke, myocardial infarction alone or following thrombolytic therapy, thermal injury, adult respiratory distress syndrome (ARDS), multiple organ injury secondary to trauma, acute glomerulonephritis, dermatoses with acute inflammatory components, acute purulent meningitis or other central nervous system disorders, Grave's disease, myasthenia gravis, scleroderma and atopic dermatitis.

The abnormal regulation and in particular the overstimulation of the NF-kB pathway correlates with inflammatory disease. The unexpected discovery that IKKα is required for globally controlling the gene expression of NF-κB-dependent genes in response to proinflammatory cytokines such as TNFα supports the use of new screening procedures to isolate compounds that diminish the severity of the inflammatory disease.

The present invention also demonstrates that biological and/or chemical agents that modulate the activity of IKKα can be used in the treatment of inflammatory disease. In particular, antagonists or inhibitors of IKKα, or of the transcription and/or translation of the IKKα gene may be employed for therapeutic and prophylactic purposes to decrease inflammation by decreasing IKKα activity in the affected tissue or organ. Antagonists of IKKα activity may be useful in ameliorating many inflammatory diseases as the term is described herein. Other methods to modulate the activity of IKKα include the use of antisense RNA or DNA targeted to the IKKα gene or regulators thereof.

The method of the invention can also be used in assays for the study of other disorders associated with activation of NF-κB unrelated to those listed above. For example, the compounds of the invention may also be useful in an assay involved in the study of cancer by enhancing the effectiveness of chemotherapeutic agents.

Examples of other diseases that can be studied using the method of the invention include hypertension, and central nervous disorders. Cells containing knockouts of regulatory genes would be appropriate candidates for the method of the invention.

Identification of Genes Involved in the NF-κB Pathway

Cell Culture

The method of the invention employs experimental cells that are deficient in a component or multiple components of the NF-κB pathway and wildtype cells. Cells that do not have a functional copy of a gene(s) for said component such as knockout cells for a component can also be used. Knockout cells can be made using techniques commonly used in the art. Hu, Y., et al. (1999) Science 284:316-320; Li, Q. et al. (1999) Genes Dev 13:1322-1328; Takeda, K., et al. Science 284:313-316. Methods for making knockout cells are known in the art and are disclosed in Gene targeting—a practical approach (2000). Second edition, Oxford University Press, incorporated herein.

Experimental and wt cells are cultured using methods known in the art in a standard growth media and standard conditions. The types of cells and tissues that can be used with the invention include cells that are capable of responding to an inflammatory agent such as mouse embryonic fibroblast (MEF) cells. Other cells that can be used include any mouse preB cells capable of responding to an inflammatory agent such as HeLa, Thp.1 and Huvec cells in which signaling components of the NF-κB activation such as macrophages and epithelial cells etc. pathway can be knockout by mutagenesis or gene silencing .

Treatment of Cells with a Stimulatory Agent

Experimental and wild type cells are exposed to a stimulatory agent. Acceptable stimulatory agents are compounds that induce expression of pro-inflammatory genes under the NF-κB pathway. Stimulatory agents include but are not limited to TNFα, IL-1 and LPS. The preferred stimulatory agent is TNFα. It is understood that other stimulatory agents that effect expression of NF-κB dependent genes can be used as well. The stimulation time and the amount of stimulatory agent that is used will vary according to the stimulatory agent used but the stimulatory agent will be administered to cells in a manner sufficient to elicit a measurable pro-inflammatory response. TNFα is added to the cells at about 1 to 10 ng/ml for 15 minutes to 24 hours. IL-1 can also be used as a stimulatory agent and can be used with about 5 to 100 ng/ml also for 15-30 minutes to up to 12 to 24 hours.

Preparation of RNA and PCR Primers

The level of gene expression can be measured by analysis of mRNA from total RNA samples. Total RNA can be prepared after delivery of the stimulating agent using methods known to those skilled in the art. Preferably total cellular RNA is isolated from tissue or cell samples using the RNeasy™ kit and Rnase-Free DNase Set Protocol from Qiagen (Valencia, Calif.) according to the manufacturer's description. Any techniques commonly used in the art for measuring the expression of a gene may be used such as northern hybridization, PCR, or dot blot analysis as described in Current Protocols in Molecular Biology, John Wiley and Sons. The level of mRNA can either be read directly or the level of a product of the mRNA such as cDNA derived from the mRNA can be measured. The level of gene expression of specific genes is compared between the experimental and wild type. Genes that have a level of expression that is modulated upward or downward relative to the wild type sample are selected and identified as genes involved in the NF-κB pathway. Genes that have a level of expression that is modified upward or downward relative to the wildtype sample are validated as target genes.

Microarray Studies

Analysis of gene expression levels can also be performed using microarray or cRNA chip analysis. These technologies allow the analysis of multiple genes in a single experiment. Preparation of cRNA, and hybridization are performed according to methods as described herein or as otherwise commonly used in the art. Microarray analysis can be performed using procedures available from various companies such as Affymetrix and Agilent technologies.

The Affymetrix procedure is the preferred method and is performed essentially as follows: Between 5 and 10 micrograms of the total RNA can be converted into double stranded cDNA by reverse transcription using a cDNA synthesis kit. The preferred kit for cDNA synthesis is Superscript Choice™ (Invitrogen, Carlsbad, Calif.), which utilizes a special oligo (dT)24 primer (Genset, La Jolla, Calif.) containing a T7 RNA polymerase promoter site added 3′ of the poly T tract. After second strand synthesis, labeled cRNA is generated from the cDNA samples by an in vitro transcription reaction using T7 RNA polymerase and a reporting reagent such as biotin-11-CTP and biotin-16-UTP (Enzo, Farmingdale, N.Y.). Other reporter agents commonly used in the art such as P³², S³⁵, fluorescein and Biotin can also be used. Labeled cRNA can be purified by techniques commonly used in the art. The preferred method is to use RNeasy spin columns (Qiagen, Valencia, Calif.). cRNA sample can be fragmented by mild alkaline treatment. Preferably, the cRNA sample is fragmented by treatment at about 94° C. for about 35 minutes in fragmentation buffer as suggested by the manufacturer. A mixture of control cRNAs for bacterial and phage genes should be included to serve as tools for comparing hybridization efficiency between arrays and for relative quantitation of measured gene expression levels. Before hybridization, the cRNA samples are heated at about 94° C. for 5 minutes, equilibrated at 45° C. for 5 minutes and clarified by centrifugation (14,000×g) at room temperature for 5 minutes. Aliquots of each cRNA sample are hybridized to arrays, according the manufacturer's directions. The arrays are washed according to as methods specified by by the manufacturer. The preferred wash is with non-stringent (6×SSPE, 0.01% Tween-20, 0.005% antifoam) and stringent (100 mm MES, 0.1M NaCl, 0.01% Tween 20), stained with R-Phycoerythrin Streptavidin-(Molecular Probes, Eugene, Oreg.), washed again and scanned by an argon-ion laser scanner with the 560-nm long-pass filter (Molecular Dynamics; Affymetrix, Santa Clara, Calif.). Data analysis can be performed in order to determine if a gene expression level is increased, decreased or unchanged. Preferably, software such as MAS 5.0 software (Affymetrix, Santa Clara, Calif.) is used.

Modulation of Gene Expression

The determination of whether a modulation of gene expression in response to a stimulatory agent has occurred is made according to the parameter as set forth. Gene expression can be modulated by either activating or increasing its expression or repressing or decreasing its expression. Modulations upward or downward are viewed with respect to the level of background inherent in the system used to measure gene expression and the level of gene expression should be distinguishable from the background levels. For instance, if the level of background typically varies two fold a modulation of gene expression of greater than two fold would be considered an increase or activation of gene expression. Generally, when gene expression is measured with a microarray apparatus, genes are selected if said gene is modulated upward or downward more than about 2 fold, preferably more than 5 fold and most preferably more than 10 fold in the experimental in a comparison to a reference control sample relative to wild type levels of expression. If modulation of gene expression is found in the experimental samples relative to the control samples for a particular gene then the gene is selected and identified as being involved in the NF-κB pathway.

Validation of Target Genes

Another embodiment of the invention provides a method for validating target genes that are involved in the NF-kB pathway. In the first step, cells that are deficient in the gene involved in the NF-kB pathway and control cells that are wild type for said gene are exposed to a stimulatory agent such that a measurable inflammatory response can be measured. The level of expression of genes implicated in the NF-kB pathway is then determined using methods commonly used in the art for measuring gene expression as otherwise described herein. Preferably measurement of gene expression is performed with a microarray apparatus. Genes are selected as targets for therapeutic intervention if the gene expression is modulated in a plurality of the NF-kb pathway implicated genes measured. Genes that have been selected using the present method are considered validated.

A number of genes known to be implicated in the NF-kB pathway can be used in the present method for validation of target genes. Candidate genes known to be implicated in the NF-kB pathway include but are not limited to the list of IL-6, IL-1a, MIP1 g, Rantes, Serum amyloid A3, and Complement component 3. In a given experiment or iteration of the method a plurality of genes implicated in the NF-kB pathway that are measured should be modulated. Preferably, greater than half of the genes measured for changes in modulation should have modulated expression in the experimental cells relative to the wt.

Genes that are identified as being involved in the NF-kB pathway using the methods described herein can be selected for validation as target genes that are involved in the NF-kB pathway and possible targets for therapeutic intervention using the present method. For instance, if gene x has been identified as being involved in the NF-kB pathway using cells deficient in a component of the NF-kB pathway, cells that have a functional copy of gene x will be used in the method for validating gene x as a target gene. Gene x will be selected as a target gene for therapeutic intervention if a plurality of NF-kB pathway implicated genes are modulated as genes for therapeutic intervention can be selected for validation.

### PREFERRED EMBODIMENT OF THE PRESENT INVENTION

Cell Culture and Treatment with Stimulatory Agent

Wild type MEFs and mutant (experimental) IKKα (−/−), IKKβ (−/−) and NEMO/IKKγ (−/−) MEFs (obtained from Dr. Michael Karin, UC San Diego) were routinely cultured in growth media (GM) consisting of DMEM, 2 mM glutamine, 10% fetal bovine serum, 100 U/ml penicillin and 100 μg/ml streptomycin. The endogenous IKK complex was stimulated by either human TNFα (10 ng/ml) (InVitrogen) or IL-1β (50 ng/ml) (Pharmingen) signaling for 2 hr or as otherwise indicated. In some experiments de novo cellular protein synthesis was inhibited by 10 min. preincubation followed by coincubation with 100 μM anisomycin (SIGMA) to block translational initiation. A trans-dominant IκBα (SS32/36AA) super repressor (IκBαSR), with serines 32 and 36 mutated to alanines was introduced into wild type MEFs by retroviral infection as previously described (Li, J., et al. (2001) J Biol Chem 276, 18579-18590). Following infection with a recombinant murine retrovirus harboring an IκBαSR-IRES-Puro expression cassette, puromycin resistant MEF populations were obtained after 6-8 days of selection in 1 μg/ml puromycin (Li, J., et al. (2001) J Biol Chem 276, 18579-18590).

Probe Preparation

Total cellular RNAs were extracted from cell lysates with an RNeasy kit (Qiagen). Purified RNAs were converted to double-stranded cDNA with a SuperScript kit (Gibco BRL) and an oligo-dt primer containing a T7 RNA polymerase promoter (GENSET). Biotin-labeled cRNAs were generated from the cDNA samples by an in vitro transcription with T7 RNA polymerase (Enzo kit, Enzo Diagnostics). The labeled cRNAs were fragmented to an average size of 35 to 200 bases by incubation at 94° C. for 35 min.

Chip Hybridization

Hybridization (16 hr), washing and staining protocols have been described {AffymetrixAffymetrix Gene Chip® Expression Analysis Technical Manual; (Mahadevappa, M., et al. (1999) Nat Biotechnol 17, 1134-1136)}. We employed Affymetrix MG-U74Av2 chips which include˜6,000 functionally characterized sequences of the murine UniGene database in addition to˜6,000 EST clusters. Chips were stained with streptavidin-phycoerythrin (Molecular Probes) and scanned with a Hewlett-Packard GeneArray Scanner.

Data Analysis

DNA microarray chip data analysis was performed using MAS 4.0 software (Affymetrix). The quantitation of each gene expressed was calculated from the hybridization intensities of 16 pairs of perfectly matched (PM) and mismatched (MM) control probe pairs with each array containing multiple internal controls for cRNA hybridization and maintenance genes (β-actin and GAPDH) for data normalization (Lockhart, D. J., et al. (1996) Nat Biotechnol 14, 1675-1680) (Affymetrix) The average of the differences (PM minus MM) for each gene-specific probe family was calculated. The software computes a variety of different parameters to determine if an RNA molecule is present or absent (Absolute Call) and whether each transcript's expression level has changed between the baseline and experimental samples (Difference Call). For a comparative chip file (such as TNFα stimulated Wt MEF vs. Wt MEF/IκBαSR), the experimental file {Wt (S)}(S=stimulated) was compared to the baseline file {IκBαSR (S)}. To minimize false positives, the following criteria were selected for significant changes for each primary screen: (1) the fold change in the average difference across all probe sets was at least 2 fold; (2) for induced genes, a difference call of “increase” or “marginal increase” should be present, and an absolute call of “presence” should be associated with the experimental file; (3) for repressed genes, a difference call of “decrease” or “marginal decrease” should be present, and an absolute call of “presence” should be associated with the baseline file. The primary software used for the selection of genes was MA4.0 and Spotfire 7.0. Hierarchical clustering was performed with the Cluster program (available at http://rana.lbl.gov/) as described previously (Eisen, M. B., et al. (1998) Proc Natl Acad Sci USA 95, 14863-14868) and all genes showing at least 2 fold changes in the primary screen Wt MEF vs. Wt MEF/IκBαSR were included. The average difference values (representing the quantity of mRNA) of the selected genes were median centered by subtracting the median observed value, normalized by genes to the magnitude (sum of the squares of the values) of a row vector to 1.0. The normalized data were clustered through one cycle of K-means clustering (K=10) and then further clustered by average linkage clustering analysis of Y axis (genes) using an uncentered correlation similarity metric, as described in the program Cluster. Average difference values of 50 or less were set to 50 before median-centering and normalization. The clustered data were visualized by the program TreeView (available at Etbb://rana.Ibl.gov/).

RT-PCRs and TaqMan Real-Time Quantitative PCR

RT-PCRs were performed as previously described (Li, J., et al. (2001) J Biol Chem 276, 18579-18590; McKenzie, F. R., et al. (2000) Mol Cell Biol 20, 2635-2649). To establish their relative qualities, serial dilutions of cDNAs were amplified with β-actin and GAPDH specific primers for internal standardization. Similarly, linear response ranges were determined for each gene to semi-quantify their expression levels. In all cases, the sizes of PCR products corresponded to those expected for each gene. PCR primer pairs were 22-24 mers as shown in sequence ID Numbers.

TaqMan Real-time quantitative PCR was based on the fluorogenic 5′ nuclease assay (Livak, K. J., et al. (1995) PCR Methods Appl 4, 357-362). The same total RNA samples that were used to prepare probes for microarray hybridization were treated with Dnase I followed by the RNeasy Mini protocol for RNA cleanup (Qiagen). The TaqMan probe consists of an oligonucleotide with a 5′-reporter dye (FAM) and a 3′-quencher dye (TAMRA). To measure the gene copy numbers of the target transcript, cloned plasmid DNA or mouse genomic DNA was serially diluted and used to produce a standard curve as described elsewhere (Li, X., et al. (2000) Brain Res Protoc 5, 211-217). Data from TaqMan PCR analyses were normalized based on mRNA copy numbers of GAPDH using the TaqMan rodent GAPDH control reagents (Applied Biosystems).

Results

IL-1 or LPS signaling via NEMO in a differentiating pre-B cell line induces a host of novel NF-κB dependent target genes but surprisingly also coordinately down-modulated a large group of genes which were also dependent on NF-κB for their repression (Li, J. et al (2001) J Biol Chem 276, 18579-18590). However, in this latter cell context we were not able to determine the individual roles of the IKKα or IKKβ signalsome subunits for the stimulation or repression of these novel NF-κB/NEMO dependent genes (Li, J., et al (2001) J Biol Chem 276, 18579-18590). Therefore, we sought an alternative biological system whereby we could determine the individual contributions of each IKK subunit for the global cellular response to NF-κB activating proinflammatory stimuli. Thus, we began a series of DNA microarray analyses with mutant MEFs genetically null for either IKKα, IKKβ or NEMO/IKKγ and examined their genomic responses to TNFα stimulation in comparison to wild type MEFs and MEFs constitutively expressing a trans-dominant IκBα super repressor mutant.

To be certain that only signalsome target genes that were dependent on NF-κB would be evaluated, we introduced a trans-dominant IκBα super repressor (IκBαSR) by retroviral transduction into Wt MEFs and performed two independent primary DNA microarray screens with and without 2 hr of TNFα stimulation. These primary screens reproducibly identified˜400 NF-κB dependent target genes that were stimulated 2 fold or more and which also exhibited average difference calls of increase in addition to appropriate present or absent absolute calls (see Methods). Up to 150 of the stimulated genes were effected 5 fold or more. Parallel secondary microarray screens comparing Wt MEFs to mutant MEFs that were null for either IKKα, IKKβ or NEMO/IKKγ were subsequently performed to determine their requirements for each signalsome subunit. As with the IκBαSR screens each of the latter IKK subunit screens were performed two independent times with excellent reproducibility. The expression status of each IKK subunit in the mutant MEF lines was verified by RT-PCR with primer pairs within the targeted exons and also by western blotting. As previously reported (Li, Z.W. et al (1999) J Exp Med 189, 1839-1845; Hu, Y. et al (1999) Science 284, 316-320; Makris, C. et al (2000) Mol Cell 5, 969-979) and as expected, the IKKα (−/−), IKKβ (−/−) and NEMO (−/−) MEF lines were only null for the expression of the targeted IKK subunit gene (data not shown).

Most NF-κB Dependent Genes Require Both IKKα and IKKβ for Their Expression

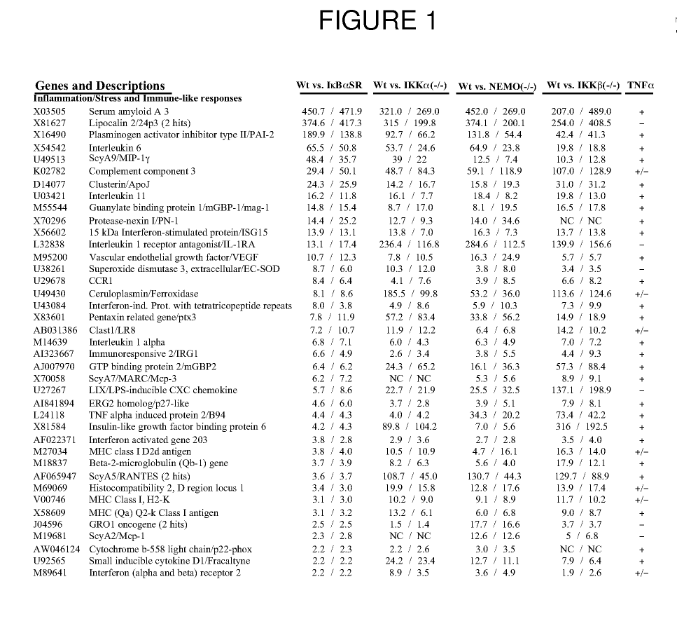

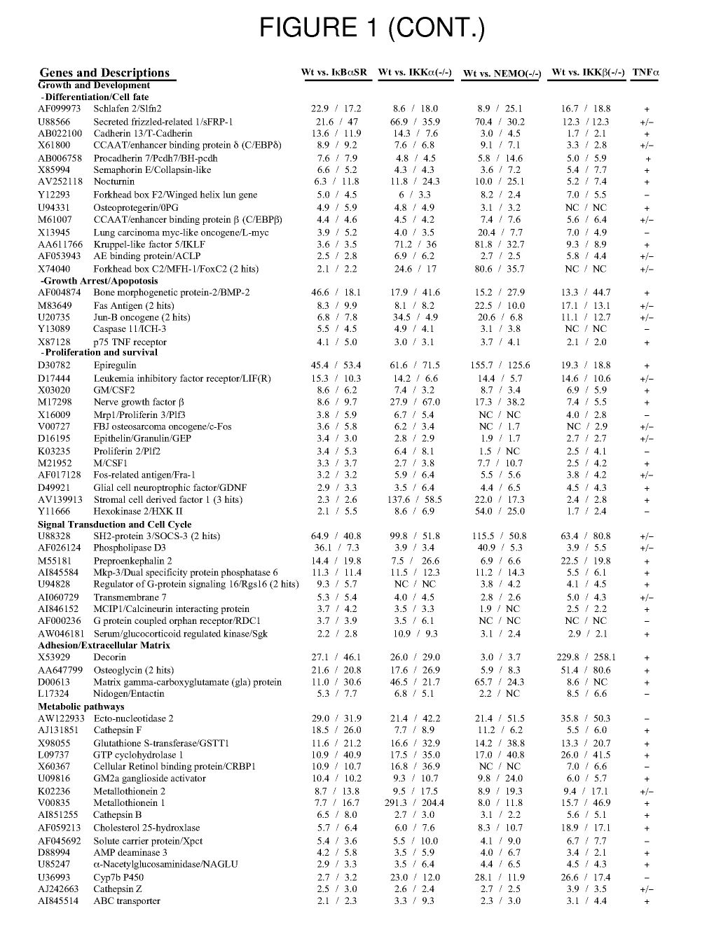

One hundred induced genes were chosen from the two primary Wt MEF vs. Wt MEF/IκBαSR screens and assembled into functional categories (see FIG. 1). FIG. 1 shows signalsome subunit requirements of selected genes in MEFs that are dependent on NF-κB for their activity. One hundred representative genes with fold change values greater than or equal to 2, that were dependent on NF-κB for maintaining their relative expression levels, were selected from a primary Wt (+TNFα) vs. IκBαSR (+TNFα) screen. Genes were grouped in categories based upon their physiological functions or properties. Gene accession numbers are in the far left column adjacent their names and descriptions. In the first data column, fold changes of genes identified in two independent primary microarray screens of Wt MEFs compared to Wt MEFs constitutively expressing the IκBα (SS32/36AA) super repressor are provided. In data columns 2-4, the dependencies of each of these genes on IKKα, NEMO/IKKγ and IKKβ were determined in six independent microarray screens wherein Wt MEFs were compared to mutant MEFs null for the individual IKK subunits. Fold change values from duplicate screenings were listed together. Dependence on one or more signalsome subunits was stringently evaluated by adhering to two criteria: wild type MEF absolute calls of “Present” (P) and “Increase” (I) average difference calls. Each screen was performed with cells stimulated for 2 hr with 10 ng/ml of human TNFα. Genes which were dependent on basal NF-κB for their expression were identified by performing independent microarray screens in the absence of TNFα stimulation. Redundancy hits (corresponding to different oligo regions of the same gene) are noted in the gene description column. NC denotes a “No Change” average difference call, indicating no significant dependence on that IKK subunit. Results of exposure to TNFα are presented in the indicated column as follows: −(no significant effect), +/−(˜2-5 fold stimulation) and +(>10 fold stimulated). Two independent screens were conducted in all cases with similar results.

A significant number of the induced genes were known NF-κB positively regulated genes including: Serum Amyloid A3, IL-6, IL-11, ISG15, IL-1RA, VEGF, Ptx3, β2 microglobulin, IL-1α, Mcp-3, RANTES, Mcp-1, Fas ligand, Jun-B, c-Fos, M/CSF and GM/CSF {reviewed in Pahl, H. L. (1999) Oncogene 18, 6853-6866}. Most of these known NF-κB dependent genes required all signalsome subunits for their activity. The activity of a synthetic NF-κB promoter driven luciferase reporter gene in each IKK null line also showed low to negligible activity in response to TNFα stimulation compared to wild type MEFs (data not shown).

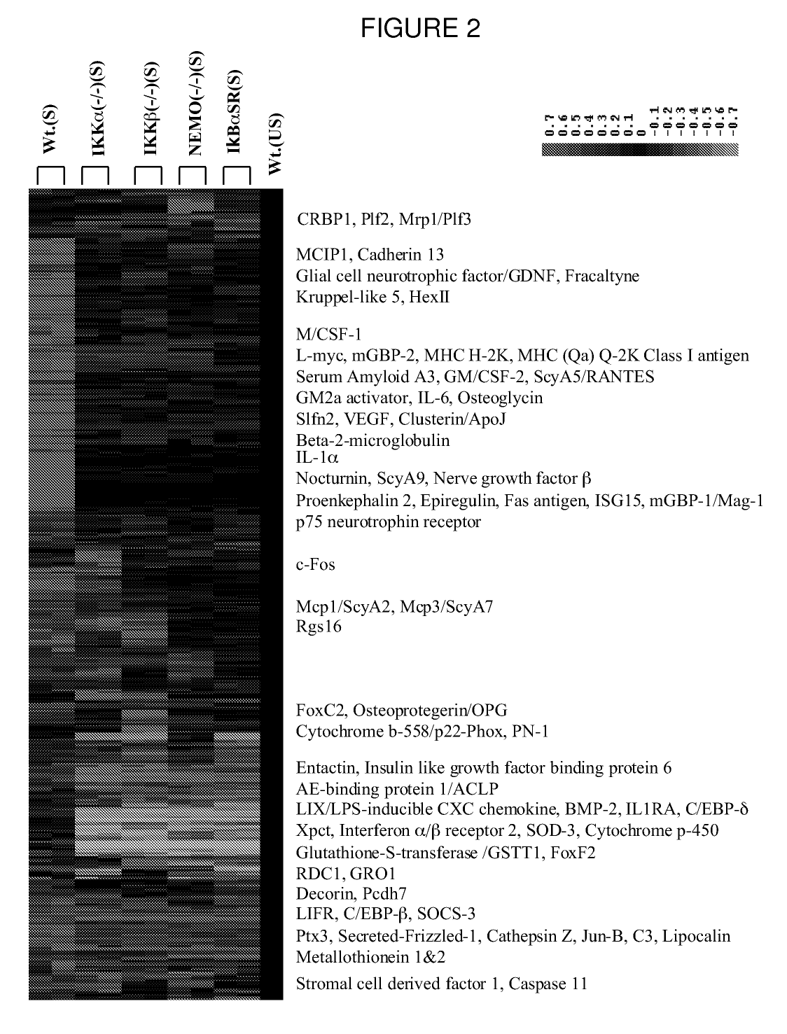

A hierarchical clustering image of all genes induced 2 fold or more in both independent microarray screens is presented in FIG. 2 which shows a hierarchical cluster image of gene expression patterns of NF-κB dependent/TNFα stimulated target genes in MEFs in the presence and absence of individual signalsome subunits. Expression profiles of all genes displaying induction of 2 fold or more and average difference calls of increase (as described in Experimental Procedures) in primary microarray screens of 2 hr TNFα stimulated Wt MEFs (lanes 1 & 2) vs. Wt MEFs constitutively expressing an IκBα (SS32/36AA) super repressor (lanes 9 & 10) were submitted to hierarchial clustering in comparison to TNFα stimulated (S) IKKα (−/−) (lanes 3 & 4), IKKβ (−/−) (lanes 5 & 6) and NEMO/IKKγ (−/−) (lanes 7 & 8) and unstimulated (US) Wt MEFs (lane 11). The locations of selected genes are indicated and their fold change values are presented in FIG. 1.

Genes labeled Wt (S) in the first two lanes of FIG. 2 were induced in response to TNFα signaling, while their expressions were inhibited in the two screens of MEFs constitutively expressing IκBαSR as noted in the ninth and tenth lanes of the figure. The eleventh and last lane of the hierarchical figure is an unstimulated (US) Wt MEF control that has been preset as described in Experimental Procedures to allow the reader to better visualize the effects on specific gene clusters. Comparisons of the first and the last lanes of FIG. 2 reveal that most genes were dependent on TNFα for the relative levels of expression to varying degrees. However a subset of genes that were dependent on basal levels of NF-κB for their activity but displayed no significant TNFα induced stimulation were also present. Surprisingly, a portion of the latter TNFα independent genes were nevertheless dependent on one or more signalsome subunits for their expression (also see FIG. 1).

Of great significance, the vast majority of the NF-κB induced genes were codependent on IKKα, IKKβ and NEMO/IKKγ for their expression with exceptional classes of genes that either required IKKα without a significant need for IKKβ and vice versa (see FIG. 1 and FIG. 2). Similar results were obtained with an independent source of wild type MEFs (data not shown). Genes whose expression levels were not significantly altered by the loss of IKKα, IKKβ or NEMO/IKKγ are listed as no change (NC) calls in FIG. 1. A small subset of NF-κB targets with the properties of IKKβ independent/IKKα dependent genes clustered together in FIG. 2 (see FoxC2, Osteprotegerin, PN-1 and Cytochrome b-558/p22-Phox). A comparably small group of probable IKKα independent/IKKβ dependent genes also clustered together (see Rgs16 and Mcp-1/ScyA2 and Mcp-3/ScyA7 in FIG. 2). A small subset of probable NEMO independent genes were also present (see CRBP1, Plf2, Mrp1/Plf3, RDC1 in FIG. 1) and three of these genes clustered together in FIG. 2 as well (see CRBP1, Plf2 and Mrp1/Plf3). This latter class of NEMO independent genes were also part of the group of TNFα independent NF-κB targets.

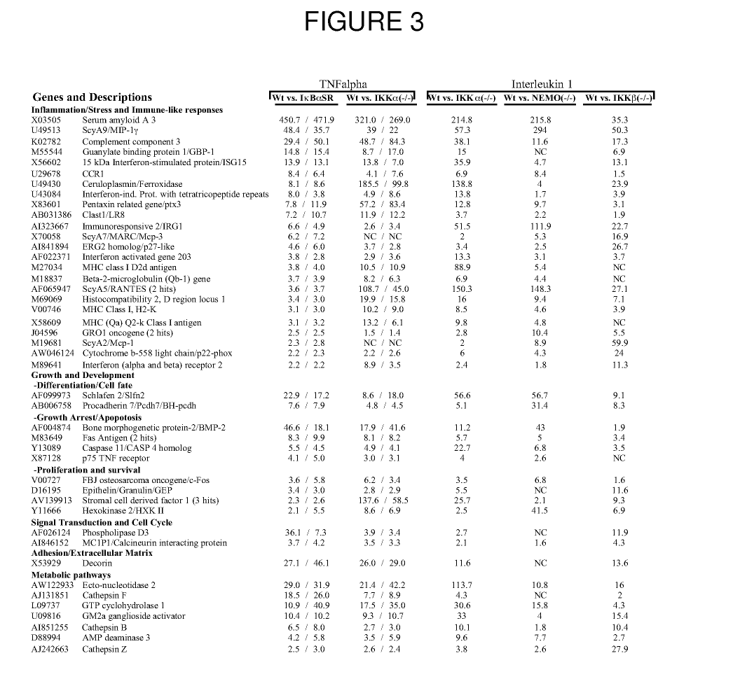

As shown in FIG. 3, three additional microarray screens revealed that 44 out of the 100 selected genes in FIG. 1 were also dependent on each IKK subunit for their response to IL-1. FIG. 3 shows signalsome subunit requirements of the selected genes in FIG. 1 for IL-1 dependent signaling Forty-four of the one hundred selected genes in FIG. 1 were found to be responsive to IL-1 signaling with similar dependencies on all signalsome subunits. Fold change values from DNA microarray screenings of IL-1 stimulated Wt MEF cells compared to IL-1 stimulated IKK subunit knockout MEF mutant cells are listed. Fold change values from the original TNFα chip screenings (FIG. 1) are included for comparison. Sixteen of the eighteen genes in FIG. 1 which were not dependent on TNFα were also not stimulated by IL-1. However, Mcp-1 and HexII, two IKK dependent genes which were not affected by TNFα, were stimulated by IL-1. In keeping with the TNFα results (see FIG. 1), the induction of Mcp-1 & 3 by IL-1 were more dependent on IKKβ and NEMO/IKKγ than IKKα (see FIG. 2). In addition, Decorin was less dependent on NEMO in the response to both TNFα and IL-1.

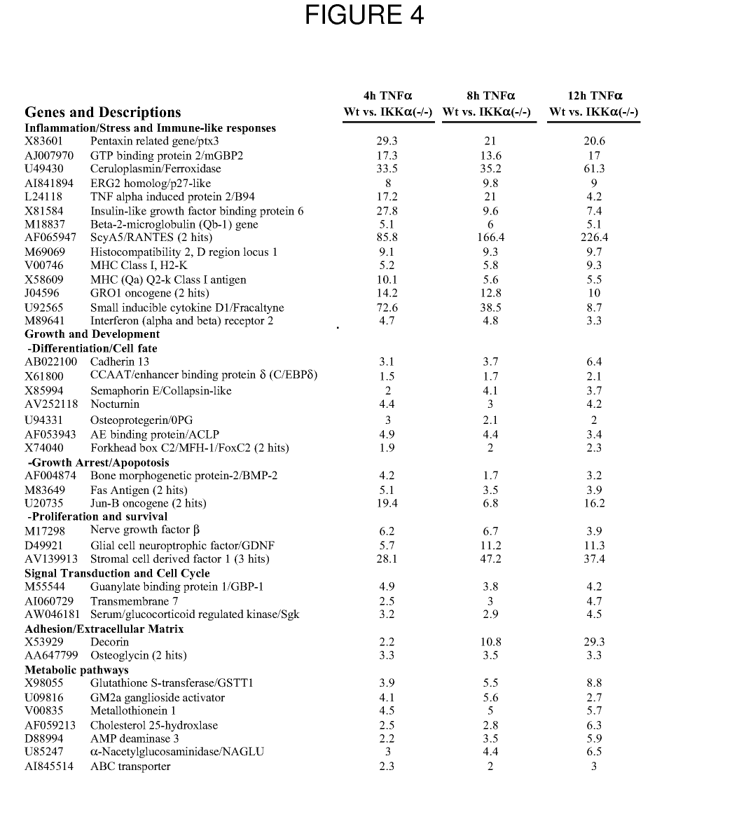

To further assess the importance of IKKα for the stimulation of NF-κB target genes by TNFα, we also performed TNFα stimulations for 4, 8 and 12 hours. As shown in FIG. 4, 39 of the 82 genes in FIG. 1, which showed evidence of TNFα inducibility, remained dependent on IKKα for their TNFα induction. FIG. 4 shows NF-κB target genes which retain their dependence on IKKα upon prolonged exposure to TNFα. Fold change values of Wt MEF cells compared to IKKα (−/−) MEFs at different time points of TNFα stimulation are listed. Thirty-nine of the eighty-two NF-κB/IKK/TNFox dependent selected genes in FIG. 1 remained dependent on IKKα after exposure to TNFα for 4, 8 and 12 hours. As discussed in the text, these 39 genes represented all of the TNFα dependent genes in FIG. 1 which retained their TNFα dependencies after prolonged exposure to the cytokine. It is also important to note that Wt (S) vs. Wt (US) comparisons showed that the 39 genes in FIG. 4 represent all of the genes in FIG. 1 which remained significantly responsive to TNFα for more than 2 hours. So in actuality none of the genes in FIG. 1 selectively lose their IKKα dependence during prolonged exposures to TNFα. Given that the NF-κB pathway is known to attenuate its own activity by inducing the expression of IκBs, a fall off in the ability of TNFα to persistently maintain the induced expression levels of a number of IKK/NF-κB target genes is not that surprising (Baldwin, A. , Jr. (1996) Annu Rev Immunol 14, 649-683; Ghosh, S., et al. (1998) Annu Rev Immunol 16, 225-260).

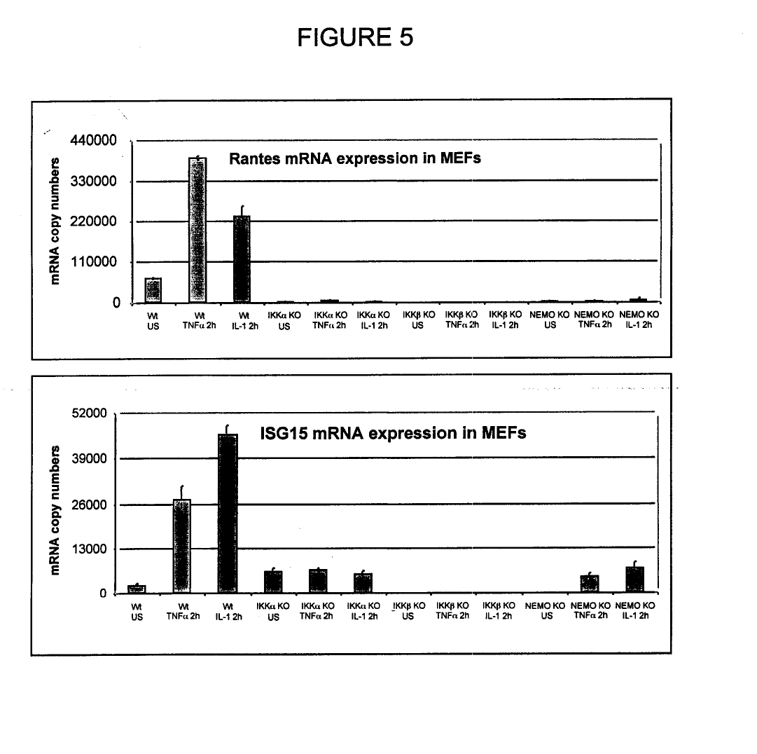

We chose several examples of genes that were codependent on NF-κB and the signalsome for their induced expression for re-examination by semi-quantitative RT-PCR or quantitative TaqMan real time PCR. TaqMan PCRs were performed for ISG15 and RANTES with and without 2 hours of TNFα or IL-1 stimulation. FIG. 5 shows TaqMan real-time PCR validations of selected induced hits from gene chip screenings. Total cellular RNAs were isolated from wild type and mutant MEFs with and without stimulation by TNFα and or IL-1 for 2 hours. RT and PCR was carried out using TaqMan quantitation (showing mRNA copy numbers detected in 40 ng total RNA). The copy numbers of gene transcripts were determined according to DNA standard and normalized with GAPDH. The Taqman primers and probes for mouse ISG15 (X56602) are SEQ. ID. No. 1 for the forward primer, SEQ. ID. No. 2 for the reverse primer and SEQ ID No. 3 for the FAM probe. The Taqman primers and probes for Rantes were provided by Applied Biosystems (Part Number: 4312879P). TaqMan PCR reactions of each individual sample were performed in triplicate, then the copy numbers and standard error were determined.

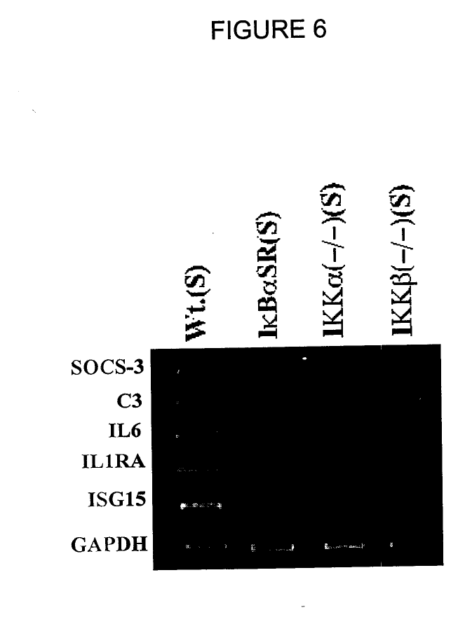

RANTES and ISG15 were strongly stimulated by either TNFα or IL-1 in wild type MEFs. However, their expression was reduced to negligible levels if not strongly inhibited in the IKKα, IKKβ or NEMO/IKKγ null cells. In keeping with the TaqMan results RT-PCRs conducted with primer pairs specific for IL-6, C3, SOCS-3, IL-1RA and ISG15 show that they are dependent on IKKα and IKKβ for their expression. FIG. 6 shows semi-quantitative RT-PCRs reveal the IKKα and IKKβ requirements of selected MEF genes within 2 hours of TNFα stimulation. All RT-PCRs were performed in the linear response range for each transcript (in comparison to a GAPDH reference control) and products were resolved on 6% PAGE and revealed by ethidium bromide staining.

Performing TNFα stimulations along with anisomycin to block translation initiation revealed that up to 50% of the TNFα dependent IKK and NF-κB dependent genes were likely to be direct targets of the NF-κB/IKK signaling pathway (data not shown). A significant portion of the NF-κB target genes in MEFs were surprisingly dependent on the IKK subunits in the absence of extracellular NF-κB activating stimuli. Examples of the latter class of genes included: PLF2 & 3, L-Myc, Caspase 11, FOXF2, RDC-1, Lipocalin, IL-1RA, Mcp-1, CRBP1, Entactin and P450 (see FIG. 1 and FIG. 4). A larger subset of genes exhibited partial dependence on TNFα signaling for their relative levels of expression but nevertheless remained extremely dependent on the IKKs in the absence of a stimulus. These latter results imply that the IKKs are required to maintain basal NF-κB activity to ensure the differential expression of specific subsets of NF-κB target genes. It is important to note in this context that constitutively activated IKKs have been observed in specific types of human lymphoid malignancies (Kordes, U. et al (2000) Leukemia 14, 399-402; Davis et al. (2001) J Exp Med 194, 1861-1874; Hinz, M. et al. (2001) Blood 97, 2798-2807). Thus, it will be important to determine in subsequent work if these IKK dependent/signal independent genes are also present in primary MEFs or whether this is a physiological property of immortalized, established cells.

Discussion

IKKα Plays a General Role in the Global Induction of NF-κB Dependent Inflammatory Response Genes

The present invention provides a method for specifically addressing the contributions of each component of the signalsome in NF-κB regulated gene expression by examining their individual effects on a host of specific NF-κB chromosomal target genes in mouse embryo fibroblasts in response to TNFα and IL-1 stimulation. It was found that IKKα is equally important as IKKβ and NEMO/IKKγ for the expression of NF-κB dependent, induced genes in these cells. Indeed many known NF-κB target genes such as IL-6, RANTES, Fas antigen, C3, Mcp-3, Ptx3, MIP-1γ, c-Fos, Serum amyloid A3, ISG15, VEGF, IL-11, IL-1α, GM/CSF2, M/CSF1, Proenkephalin, GRO1, β2 Microglobulin and several other MHC molecules were not stimulated by TNFα nor IL-1 in the absence of IKKα. This demonstrates an unexpected role for IKKα in the global control of NF-κB-dependent gene expression in response to two major inflammatory response cytokines. Indeed, the largest subset of NF-κB dependent genes were those involved in inflammatory, stress or immune-like responses which exhibited a strong codependency on IKKα and IKKβ with few exceptions (see FIG. 1 and FIG. 2). A small number of the NF-κB dependent chromosomal targets also revealed preferential dependencies on IKKα or IKKβ, indicating that their roles in NF-κB activation appear to be target gene dependent in the same cellular background and in response to the same extracellular signal. In addition, the unexpected presence of NF-κB/IKK dependent, signal independent genes suggests that the signalsome may also play a role in maintaining the activities of genes regulated by basal levels of activated NF-κB.

Even though it is well established that IKKβ is essential for the release of NF-κB from IκB and the subsequent acquisition of NF-κB DNA binding activity (Karin, M. (1999) Oncogene 18, 6867-6874; Li, Q., et al (1999) Science 284, 321-325; Li, Z., et al. (1999) J Exp Med 189, 1839-1845; Tanaka, et al. (1999) Immunity 10, 421-429; Delhase, M., et al. (1999) Science 284, 309-313; Karin, M., and Ben-Neriah, Y. (2000) Annu Rev Immunol 18, 621-663 and Baud, V., et al. (1999) Genes Dev 13, 1297-1308), a number of studies have indicated the possibility of additional levels of control in the cytokine and IKK mediated control of NF-κB activation that are independent of its liberation from IκB. Inhibitors of phosphatidylcholine specific phospholipase C and protein kinase C were initially reported to block the activation of NF-κB by TNFα and IL-1 signaling without effecting IκBα degradation or NF-κB DNA binding activity (Bergmann, et al. (1998) J Biol Chem 273, 6607-6610). Subsequently, similar results were reported for the mechanism of phosphatidylinositol-3-OH kinase (PI3K) and PI3K-activated kinase B/Akt dependent NF-κB activation by IL-1 signaling, which was shown to involve the phosphorylation of the RelA/p65 activation domain (Sizemore, N., et al. (1999) Mol Cell Biol 19, 4798-4805). Several other studies suggesting that NF-κB transcriptional competence was regulated independent of IκB revealed that the catalytic subunit of protein kinase A (PKAc) phosphorylated RelA/p65 thereby facilitating its binding to the transcriptional co-activators CREB binding protein (CBP) and its p300 homolog (Gerristen, et al. (1997) Proc Natl Acad Sci USA 94, 2927-2932; Perkins, N. D., et al. (1997) Science 275, 523-527; Zhong, et al. (1997) Cell 89, 413-424; Zhong, H., Voll, R. E., and Ghosh, S. (1998) Mol. Cell. 1, 661-671). TNFα induced p65/RelA transactivation was blocked by specific inhibitors of the p38 stress and mitogen-activated protein kinases (MAPK) (Vanden Berghe, W., et al. (1998) J Biol Chem 273, 3285-3290) and TNFα mediated p65 phosphorylation was localized to serine 529 within the p65 transcriptional activation domain (TAD) (Wang, D. et al (1998) J Biol Chem 273, 29411-29416). Multiple serines within c-Rel's carboxy-proximal TAD were subsequently shown to be necessary for TNFα-induced c-Rel activation; and PI3K and ζPKC were also identified as two putative downstream effectors, whose activities were both necessary for c-Rel transactivation activity (Martin, A. G., et al (2000) J Biol Chem 275, 24383-24391; Martin, A. G. et al (2001) J Biol Chem 276, 15840-15849). Continuing along the same theme, PI3K- and Akt-dependent signaling pathways were reported to stimulate the p65 TAD via IKKβ and were also functionally and mechanistically correlated with Akt's anti-apoptotic activity (Madrid, L. V. et al (2000) Mol Cell Biol 20, 1626-1638). RelA/p65 serine 536 was subsequently shown to be phosphorylated by IKKβ in vitro and in vivo (Mercurio, F. et al (1997) Science 278, 860-866; Sakurai, H. et al (1999) J Biol Chem 274, 30353-30356). The molecular requirements for Akt mediated activation of the p65 TAD were further dissected to reveal that: (a) p65 TAD serines 529 and 536 were both required by Akt signaling, which operated at least in part via IKKβ; and (b) Akt and IL-1 signaling also activated p38 in an undefined IKKα dependent pathway, which appeared in part to facilitate p65 engagement with the CBP/p300 co-activator (Madrid, L. V. et al (2001) J Biol Chem 276, 18934-18940). Akt activation in vivo requires PIP3 (phosphatidylinositol 3,4,5-triphosphate), a natural product of PI3K activity; and PIP3 is down-regulated by PTEN, a lipid phosphatase and tumor suppressor (Cantley, L. C. et al (1991) Proc Natl Acad Sci USA 96, 4240-4245). PTEN was initially reported to inhibit TNFα induced NF-κB transactivation and DNA binding activity (Koul, D. et al (2001) J Biol Chem 276, 11402-11408; Gustin, J. A. et al (2001) J Biol Chem 276, 27740-27744). However, a more recent study showed that PTEN only inhibited p65 transactivation and not NF-κB DNA binding, which was rescued by over-expression of activated forms of PI3K, Akt or Akt and IKK providing additional support for the controversial role of the PI3K-Akt pathway in uniquely controlling NF-κB transactivation potential (Mayo, M. W. et al (2002) J Biol Chem 277, 11116-11125). Interestingly, more recent efforts have shown that efficient IL-1 and Akt mediated NF-κB transactivation appears to require both IKKα and IKKβ, which are also codependent on each other for p65 TAD phosphorylation (Sizemore, N. et al (2002) J Biol Chem 277, 3863-3869). Taken together, the above findings reveal that important gaps remain in our knowledge regarding the physiological significance and controversial mechanisms of action of the IKK complex for establishing the transcriptional competence of DNA bound NF-κB. Furthermore, our findings show that IKKα plays an unexpectedly general role in the global competence of NF-κB and indicate that IKKα is likely to be a direct or indirect contributor to a number of these phenomenon.

Novel NF-κB Dependent Genes Encoding Regulators of Gene Expression, Differentiation and Cellular Fate

Positive and Negative Effectors of Cellular Proliferation and Mortality Were amongst the Genes Dependent on IKK/NF-κB Signaling

Genes encoding proteins that directly influence cellular growth required the IKKs and NF-κB for their expression. Epiregulin (an EGF-like autocrine growth factor for kerotinocytes) (Shirakata, Y. et al (2000) J Biol Chem 275, 5748-5753), Granulin/epithelin precursor/GEP (a potent MEF specific growth factor that functions independent of insulin-like growth factor receptor) (Zanocco-Marani, T. et al (1999) Cancer Res 59, 5331-5340), Stromal cell derived growth factor (a potent lymphocyte chemotactic chemokine activity produced by stromal cells) (Bleul, C. C. et al (1996) J Exp Med 184, 1101-1109) and Leukemia inhibitory factor receptors (Tanaka, M. et al (1999) Blood 93, 804-815) were amongst the TNFa responsive IKK dependent genes. However, Proliferin 2/PLF2 and Proliferin 3/PLF3/Mrp1 (Groskopf, J. C. et al (1997) Endocrinology 138, 2835-2840; Toft, D. J. et al (2001) Proc Natl Acad Sci USA 98, 13055-13059), which are prolactin related hormones with angiogenic properties which also stimulate endothelial cell chemotaxis, belonged to a subset of genes which were dependent on IKKα and IKKβ in the absence of TNFα stimulation in MEFs. Several neurotrophic activities including the p75 neurotrophin receptor, nerve growth factor β and glial cell-derived neurotrophic factor (GDNF) were also dependent on the IKKs and TNFα signaling for their levels of expression and p75 was also responsive to IL-1 signaling.

Several negative effectors of cellular growth, cell cycle progression, cellular viability or inflammatory reactions were also surprisingly dependent on IKKα and IKKβ for their TNF stimulation including Clusterin/ApoJ, BMP-2 and Schlafen 2 and the p75 neurotrophin receptor, while Caspase 11 appeared to be dependent on IKKα and NEMO in the absence of extracellular NF-κB stimuli. BMP-2 has been reported to promote apoptosis in a SMAD independent, protein kinase C dependent pathway by increasing the Bax/Bcl-2 ratio and increasing the release of cytochrome C from mitochondria. Enforced expression of Schlafen 2 in transgenic mice has been reported to block double positive thymocyte maturation and to retard fibroblast cell growth in vitro (Schwarz, D. A. et al (1998) Immunity 9, 657-668). Nerve growth factor has been reported to illicit pro-apoptotic effects on neuroblastoma cells via the p75 neurotrophin receptor, while it can also promote a survival response upon signaling via the homologous TrkA neurotrophin receptor (Bono, F., (1999) FEBS Lett 457, 93-97). The p75NTR has also been shown to promote apoptosis by binding to beta amyloid peptide, an effect which is enhanced by IL-1 signaling (Perini, G. et al (2002) J Exp Med 195, 907-918). Because IKK mediated NF-κB activation by TNFα has been shown to promote neuronal cell survival (Mattson, M. P. (2000) J Neurochem 74, 443-456), subsequent activation of p75 by NF-κB could also be a double edged sword, contributing under some physiological situations to apoptotic responses. Caspase 11/Ich-3 is a member of the ice/ced family of death promoting proteins (Wang, S. et al(1996) J Biol Chem 271, 20580-20587). It is dramatically induced by mediators of septic shock and promotes apoptosis, which can be abrogated by the Bcl-2 survival factor (Wang, S. et al Biol Chem 271, 20580-20587). Therefore, under some physiological circumstances IKK mediated NF-κB activation can have unexpected inhibitory effects on cellular growth, cell cycle progression and cellular viability.

Clusterin/ApoJ, a molecular chaperone-like glycoprotein, has been well documented to accumulate at the sites of tissue remodeling and degeneration in various disease states (Silkensen, J. R. et al (1994) Biochem Cell Biol 72, 483-488; Rosenberg, M. E., and Silkensen, J. (1995) Int J Biochem Cell Biol 27, 633-645). ApoJ was also repressed in proliferating cells and its over-expression was recently shown to impede cell cycle progression of transformed cells in vitro (Silkensen, J. R. et al (1994) Biochem Cell Biol 72, 483-488; Rosenberg, M. E. et al (1995) Int J Biochem Cell Biol 27, 633-645). Clusterin/ApoJ was also recently reported to act in an anti-inflammatory capacity in vivo by regulating immune complex metabolism and clearance with ApoJ deficient mice exhibiting enhanced kidney aging due to immune complex deposition (Rosenberg, M. E. et al (2002) Mol. Cell. Biol. 22, 1893-1902). Therefore, induction of Clusterin/ApoJ by NF-κB could conceivably protect against immune complex mediated inflammatory reactions in vivo.

Important Regulators of Signal Transduction and Metabolic Pathways Requiring NF-κB/IKK Signaling for their Expression

Components of NF-κB independent signal transduction and metabolic pathways were also amongst the novel target genes downstream of IKK mediated NF-κB activation. SOCS-3, a negative regulator of STAT3 signaling (Starr, R. et al (1997) Nature 387, 917-921), was a TNFα dependent NF-κB/IKK target, revealing a novel type of regulatory cross-talk wherein NF-κB has the potential to simultaneously inhibit STAT signaling pathways. SOCS-3 was also recently shown to be an intracellular effector of IL-10 induced anti-inflammatory responses in macrophages, where it was capable of blocking the LPS induced expression of a number of NF-κB target genes including IL-6, TNFα and GM-CSF (Berlato, C. et al (2002) J Immunol 168, 6404-6411). Consequently, the activation of SOCS-3 by NF-κB/IKK could conceivably represent a novel mechanism to attenuate NF-κB induced inflammatory responses. In addition to SOCS-3, intracellular effectors of other NF-κB independent signaling pathways were also found to be NF-κB/IKK dependent. GBP1/Mag-1 and mGBP2, 65-kDa GTPases which were known to be amongst the genes activated in the cellular response to IFN-γ (Wynn, T. A. et al (1991) J Immunol 147, 4384-4392; Boehm, U. et al (1998) J Immunol 161, 6715-6723), were both found to be strongly dependent on NF-κB and each IKK for its induction by TNFα and mGBP1/Mag-1 was also dependent on IKKα for its stimulation by IL-1. Interestingly, Interferon (alpha and beta) receptor 2 (the murine homolog of the human interferon alpha receptor) was also found to be dependent on IKKα and IKKβ for its TNFα stimulation (Uze, G. et al (1992) Proc Natl Acad Sci USA 89, 4774-4778). Rgs16, a negative regulator of G-protein-coupled receptor (GPCR) signaling induced in response to bacterial infection (Beadling, C. et al (1999) J Immunol 162, 2677-2682; Panetta, R. et al (1999) Biochem Biophys Res Commun 259, 550-556) that we had previously shown to be dependent on NEMO and NF-κB in a differentiating pre-B cell line (Li, J. et al (2001) J Biol Chem 276, 18579-18590) was dependent on NEMO but not significantly on either IKKα or IKKβ in MEFs. RDC1, an orphan G-protein coupled receptor and a novel HIV/SIV co-receptor (Shimizu, N. et al (2000) J Virol 74, 619-626), was dependent on NF-κB and IKKα for its TNFα independent expression but did not appear to be independent of NEMO nor IKKβ. MCIP1, (myocyte-enriched calcineurin interacting protein) which is located in the Down syndrome critical region and can act as a blocker of calcineurin signaling (Rothermel, B. et al (2000) J Biol Chem 275, 8719-8725), was dependent on IKKα and IKKβ for its TNF induced expression. The 22-kDa subunit of Cytochrome b-558/p22-Phox, an essential component of the phagocytic NADPH-oxidase responsible for superoxide generation and absent in inherited chronic granulomatous disease (CGD) (Parkos, C. A. et al (1988) Proc Natl Acad Sci USA 85, 3319-3323; Dinauer, M. C. et al (1990) J Clin Invest 86, 1729-1737), was dependent on IKKα but not IKKβ for its stimulation by TNFα while it was dependent on both catalytic IKKs for its activation by IL-1 signaling. Ceruloplasmin/Ferroxidase, a copper and iron binding oxidoreductase which is upregulated in acute phase inflammatory responses (Aldred, A. R. et al (1987) J Biol Chem 262, 2875-2878; Klomp, L. W. et al (1996) J Clin Invest 98, 207-215), was highly dependent on the IKKs for its expression and stimulation by TNFα and IL-1. NAGLU (α-Nacetylglucosaminidase), which is required for heparin sulfate degradation and known to be responsible for the rare autosomal recessive disorder Sanfilippo syndrome (Aldred, A. R. et al (1987) J Biol Chem 262, 2875-2878; Klomp, L. W., Farhangrazi et al 1996) J Clin Invest 98, 207-215), required IKKα and IKKβ for its response to TNFα. In addition, the G_{M2} activator, which plays an essential role in the lysosomal degradation of G_{M2} gangliosides and is the causal deficiency of neurodegenerative Tay-Sachs and Sandhoff diseases (Liu, Y., Hoffmann, A. et al (1997) Proc Natl Acad Sci USA 94, 8138-8143), required each IKK for its response to TNFα and IL-1. Cholesterol 25-hydroxylase, which synthesizes 25-hyroxycholesterol (a co-repressor that reduces cholesterol biosynthesis and blocks sterol regulatory element binding protein processing) (Lund, E. G. et al J Biol Chem 273, 34316-34327), was amongst the IKK/NF-κB/TNFα dependent genes. Coordinate TNFα induction of Decorin and Osteoglycin, two members of the lecuine rich repeat family of proteoglycans (Matsushima, N. et al al (2000) Proteins 38, 210-225), was also dependent on each IKK subunit; and Decorin was also responsive to IL-1 signaling. Finally, three Cathepsin cysteine proteinases (Cathepsins B, F and Z) (Qian, F. et al (1991) DNA Cell Biol 10, 159-168; Santamaria, I. et al (1998) J Biol Chem 273, 16816-16823; Santamaria, I. (1999) J Biol Chem 274, 13800-13809) were also coordinately dependent on IKKα and IKKβ for their stimulation by TNFα and IL-1.

### SUMMARY

The IKKβ and NEMO/IKKγ subunits of the NF-κB activating signalsome complex are known to be essential for activating NF-κB by inflammatory and other stress-like stimuli. However, the IKKα subunit is believed to be dispensable for the latter responses and instead functions as an in vivo mediator of other novel NF-κB dependent and independent functions. In contrast to this generally accepted view of IKKα's physiological functions, we demonstrate in mouse embryonic fibroblasts (MEFs) that, akin to IKKβ and NEMO/IKKγ, IKKα is also a global regulator of TNFα and IL-1 responsive IKK signalsome-dependent target genes including many known NF-κB targets such as Serum amyloid A3, C3, IL-6, IL-11, IL-1RA, VEGF, Ptx3, β2 microglobulin, IL-1α, Mcp-1 & 3, RANTES, Fas antigen, Jun-B, c-Fos, M/CSF and GM/CSF. Only a small number of NF-κB dependent target genes were preferentially dependent on IKKα or IKKβ. Constitutive expression of a trans-dominant IκBα super repressor (IκBαSR) in wild type MEFs confirmed that these signalsome dependent target genes were also dependent on NF-κB. A subset of NF-κB target genes were IKK dependent in the absence of exogenous stimuli suggesting that the signalsome was also required to regulate basal levels of activated NF-κB in established MEFs. Overall, a sizeable number of novel NF-κB/IKK dependent genes were identified including Secreted Frizzled, Cadherin 13, Protocadherin 7, C/EBPβ & δ, Osteoprotegerin, FOXC2 & F2, BMP-2, p75 neurotrophin receptor, Guanylate binding proteins 1 and 2, ApoJ/Clusterin, Interferon (α & β) receptor 2, Decorin, Osteoglycin, Epiregulin, Proliferins 2 & 3, Stromal Cell derived factor and Cathepsins B, F and Z. SOCS-3, a negative effector of STAT3 signaling was found to be an NF-κB/IKK induced gene, suggesting that IKK mediated NF-κB activation can coordinately illicit negative effects on STAT signaling.

We claim:

1. A method for identifying genes involved in the NF-κB pathway comprised of the steps of:
a. determining the level of expression of a gene in an experimental sample obtained from cells having deficient levels of a component of the NF-κB pathway wherein the cells have been exposed to a stimulatory agent;
b. determining the level of expression of said gene in a control sample obtained from wild type cells having wild type levels of a component of the NF-κB pathway wherein the wild type cells have been exposed to said stimulatory agent;
c. selecting genes having a level of expression that is modulated upward or downward in said experimental sample relative to said wild type sample.

2. The method of claim 1 wherein the stimulatory agent is TNFα or IL-1.

3. The method of claim 1 wherein the cells are MEF cells.

4. The method of claim 1 wherein the component of the NF-κB pathway is selected from, IKKα, IKKβ and NEMO/IKKγ.

5. The method of claim 4 wherein the component is IKKα.

6. The method of claim 1 wherein the cells of said experimental sample are knockout cells having a −/− genotype for a component of the signalsome.

7. The method of claim 1 wherein genes are selected if said gene is modulated upward or downward with respect to the level of background inherent in the system used to measure gene expression and the level of gene expression.

8. The method of claim 1 wherein the level of gene expression is determined by analysis of the levels of gene expression with a microarray apparatus.

9. The method of claim 1 wherein genes are selected if said gene is modulated upward or downward more than about 2 fold in control relative to wild type levels of expression.

10. The method of claim 9 wherein genes are selected if said gene is modulated upward or downward more than about 5 fold in control relative to wild type levels of expression.

11. The method of claim 10 wherein genes are selected if said gene is modulated upward or downward more than about 10 fold in control sample relative to wild type levels of expression.

12. A method for validating target genes of:
a. exposing experimental cells deficient in a gene involved in the NF-κB pathway and control cells that are wild type for said gene to a stimulatory agent;
b. determining the level of expression of NF-κB implicated genes;
c. selecting genes as targets for therapeutic intervention if a plurality of the implicated genes are modulated.

13. The method of claim 12 wherein the cells are deficient in IKKα, IKKβ and NEMO/IKKγ.

14. The method of claim 12 wherein said cells have a gene knockout for IKKα, IKKβ or NEMO.

15. The method of claim 12 wherein the cells are deficient in IKKα.

16. A method of treating inflammatory disease by modulating the expression of genes identified as being under the control of IKKα using the method of claim 1.

17. A method of treating inflammatory disease by administering to a patient needing such treatment a therapeutically effective amount of a modulator of the expression of a gene selected from the list consisting of decorin, protease-nexin, ISG15, ERG2, G protein coupled receptor RDC1, glucocorticoid-regulated kinase (SGK), phiospholipase D3, hexokinase 2, and Mkp-3/Dual specific protein phosphatase 6, ABC transporter Fox/Forkhead, members of the Frizzled family of Wnt signaling receptors, C-EBPβ and C/EBPγ homologous transcriptional regulators of inflammatory responses and SOCS-3.

18. A method of treating inflammatory disease by administering to a patient needing such treatment a therapeutically effective amount of a modulator of the activity of the gene products of genes identified as being under the control of IKKα.

19. The method of claim 18 wherein said gene products are selected from decorin, protease-nexin, ISG15, ERG2, G protein coupled receptor RDC1, glucocorticoid-regulated kinase (SGK), phiospholipase D3, hexokinase 2, and Mkp-3/Dual specific protein phosphatase 6, ABC transporter, Fox/Forkhead, members of the Frizzled family of Wnt signaling receptors, C-EBPβ and C/EBPγ homologous transcriptional regulators of inflammatory responses and SOCS-3.

20. A method of treating inflammatory related diseases by administering to a patient in needing such treatment a therapeutically effective amount of a modulator of the activity of IKKα.
